(19) United States
(12) Patent Application Publication
(10) Pub. No.: US 2008/0103118 A1
(43) Pub. Date: May 1, 2008

(54) COMPOSITION FOR IMPROVING BLOOD CHOLESTEROL LEVELS

(75) Inventors: Ken Clement, Mississauga (CA); Marvin A. Heuer, Mississauga (CA); Megan Thomas, Mississauga (CA); Erin Mason, Mississauga (CA)

Correspondence Address: TORYS LLP, 79 WELLINGTON ST. WEST, SUITE 3000, TORONTO, ON, M5K 1N2, US

(73) Assignee: IOMEDIX DEVELOPMENT INTERNATIONAL SRL, St. Michael (BB)

(21) Appl. No.: 11/867,495

(22) Filed: Oct. 4, 2007

Related U.S. Application Data
(60) Provisional application No. 60/863,163, filed on Oct. 27, 2006.

(51) Int. Cl. A61K 31/56; A61P 9/00

(52) U.S. Cl. 514/169

(57) ABSTRACT

A nutritional composition for improving blood cholesterol by jointly and simultaneously inhibiting cholesterol absorption, decreasing blood LDL levels, increasing blood HDL levels and interfering with HMG-CoA reductase synthesis or degradation in an individual comprising, therapeutically effective amounts of plant sterols or plant stanols or derivatives thereof, procyanidins, policosanol and niacin or derivatives of niacin is provided. Both a composition and a method are provided by the present disclosure.

### RELATED APPLICATIONS

The present application is related to and claims benefit of priority to U.S. Provisional Application No. 60/863,163 entitled “Composition for improving blood cholesterol levels” filed Oct. 27, 2006, the disclosure of which is hereby fully incorporated by reference.

### FIELD OF THE INVENTION

The present invention is related to nutritional compositions for improving blood cholesterol levels in an individual. More specifically, the present invention relates to a nutritional composition comprising a combination of plant sterols or derivatives of plant sterols, procyanidins, policosanol and niacin or derivatives of niacin. An additional aspect of the present invention relates to a nutritional composition comprising a combination of plant stanols or derivatives of plant stanols, procyanidins, policosanol and niacin or derivatives of niacin.

### BACKGROUND OF THE INVENTION

The serum lipid profile is used to assess the risk an individual has for cardiovascular disease (Brehm A, Pfeiler G, Pacini G, Vierhapper H, Roden M. Relationship between serum lipoprotein ratios and insulin resistance in obesity. Clin Chem. 2004 December; 50(12):2316-22). Among the various parameters measured are the levels of triglycerides, total cholesterol, LDL-cholesterol, and HDL-cholesterol.

Low density lipoproteins (LDL) are considered to be the unhealthy type of cholesterol, whereas high density lipoproteins (HDL) are considered to be the healthy type of cholesterol. High levels of serum HDL have long been associated with good health (Ullman K. HDL Becoming Important Piece of CHD Puzzle. DOC News. February 2006; 3:9). While cholesterol is essential for cell membranes in addition to being a precursor for bile acid and steroid hormone synthesis, it is poorly soluble in blood and requires the assistance of transport molecules. Lipoproteins provide this function to act as vehicles for the transport of cholesterol. In addition to the specific proteins of which HDL and LDL are comprised, they also differ in size and density. An HDL is the smallest lipoprotein and is largely involved in the removal of excess cholesterol, which may be disposed of in the liver (Barter P. The role of HDL-cholesterol in preventing atherosclerotic disease. Eur Heart J Suppl. 2005 May; (Suppl F): F4-F8). LDL on the other hand are larger than HDL and are the main transporter of cholesterol within the blood. Blood transports cholesterol to cells for use, including the arteries, where high levels of cholesterol may lead to the formation of plaques resulting in cardiovascular disease. One of the most accurate and accepted predictors of health measures is the HDL/LDL ratio (Brehm A, Pfeiler G, Pacini G, Vierhapper H, Roden M. Relationship between serum lipoprotein ratios and insulin resistance in obesity. Clin Chem. 2004 December; 50(12):2316-22). Body weight reduction, through dieting, has been shown to favorably change this ratio (Roberts C K, Barnard R J. Effects of exercise and diet on chronic disease. J Appl Physiol. 2005 January; 98(1):3-30).

Cholesterol used by the body is either obtained from the diet or synthesized by the body. Cholesterol is primarily synthesized through the 3-hydroxy-3-methylgluteryl CoA (HMG-CoA) reductase pathway. HMG-CoA reductase is considered to be the rate limiting step in the biosynthesis of cholesterol (Kleemann R, Kooistra T. HMG-CoA reductase inhibitors: effects on chronic subacute inflammation and onset of atherosclerosis induced by dietary cholesterol. Curr Drug Targets Cardiovasc Haematol Disord. 2005 December; 5(6):441-53). Inhibition of the HMG-CoA reductase enzyme has been shown to be a viable and effective therapy for treating and preventing coronary heart disease by lowering cholesterol levels (van Hout B A, Simoons M L. Cost-effectiveness of HMG coenzyme reductase inhibitors; whom to treat? Eur Heart J. 2001 May; 22(9):751-61).

### SUMMARY OF THE INVENTION

The present invention is directed towards a nutritional composition comprising an effective amount of plant sterols or derivatives of plant sterols, a source of an effective amount of procyanidins, a source of an effective amount of policosanol, and an effective amount of niacin or derivatives of niacin. The ingredients of the present composition act substantially simultaneously to promote improved blood cholesterol levels by inhibiting cholesterol absorption, decreasing total blood cholesterol levels, decreasing blood LDL levels, increasing blood HDL levels and interfering with HMG-COA reductase synthesis while facilitating its degradation. Both a composition and a method are provided by the present disclosure.

In an additional embodiment of the present the nutritional composition comprises an effective amount of plant stanols or derivatives of plant stanols, a source of an effective amount of procyanidins, a source of an effective amount of policosanol, and an effective amount of niacin or derivatives of niacin. The ingredients of the present composition act substantially simultaneously to promote improved blood cholesterol levels by inhibiting cholesterol absorption, decreasing total blood cholesterol levels, decreasing blood LDL levels, increasing blood HDL levels and interfering with HMG-CoA reductase synthesis while facilitating its degradation. Both a composition and a method are provided by the present disclosure.

In various embodiments, the method and composition may comprise multi-phasic dissolution characteristic of the ingredients, providing time-release mechanisms.

### DETAILED DESCRIPTION OF THE INVENTION

In the following description, for the purposes of explanations, numerous specific details are set forth in order to provide a thorough understanding of the present invention. It will be apparent, however, to one skilled in the art that the present invention may be practiced without these specific details.

The present invention is directed towards a nutritional composition for improving blood cholesterol levels in an individual by acting substantially simultaneously to inhibit cholesterol absorption, decrease total blood cholesterol levels, decrease blood LDL levels, increase blood HDL levels and interfere with HMG-CoA reductase synthesis while facilitating its degradation.

Derivatives of plant sterols and plant stanols refer to any plant sterol or plant stanol resulting from chemical modification. In particular, derivatives of plant sterols and plant stanols include plant sterol esters and plant stanol esters. The esterification of plant sterols and stanols is known in the food industry and is practiced to increase the solubility of the sterols and stanols for inclusion in foodstuffs such as margarine (Law M. Plant sterol and stanol margarines and health. BMJ. 2000 Mar. 25; 320(7238):861-4).

It is herein understood that improved blood cholesterol levels may be mediated by multiple, non-mutually exclusive mechanisms including but not limited to reduction of cholesterol absorption, reduction of blood cholesterol levels, reduction of cholesterol synthesis, reduction of the blood levels of LDL and increases in the blood levels of HDL.

Furthermore, it is understood that improved blood cholesterol levels may be mediated, in part, by interference with the synthesis of endogenous cholesterol in an individual through interference with the activity of biosynthetic enzymes responsible for cholesterol synthesis such as HMG-CoA reductase. It is further understood that interference with the activity of HMG-CoA reductase may be achieved via mechanisms including but not limited to transcription, translation, post-translational modifications, protein degradation and enzymatic activity of mature proteins.

As used herein, the term ‘nutritional composition’ includes dietary supplements, diet supplements, nutritional supplements, supplemental compositions and supplemental dietary compositions or those similarly envisioned and termed compositions not belonging to the conventional definition of pharmaceutical interventions as is known in the art. Furthermore, ‘nutritional compositions’ as disclosed herein belong to category of compositions having at least one physiological function when administered to a mammal by conventional routes of administration.

Alternatively, formulations and nutritional compositions belonging to the present invention may be considered to be nutraceuticals. As used herein, the term ‘nutraceutical’ is recognized and used in the art to describe a specific chemical compound or combination of compounds found in, organic matter for example, which may prevent, ameliorate or otherwise confer benefits against an undesirable condition. As is known in the art, the term ‘nutraceutical’ is used to refer any substance that is a food, a part of food, or an extract of food which is suitable for consumption by an individual and providing physiological benefit which may be medical or health-related. Furthermore, the term has been used to refer to a product isolated, extracted or purified from foods or naturally-derived material suitable for consumption by an individual and usually sold in medicinal forms, such as caplets, tablet, capsules, soft-gel™ caplets, gel-caps and the like, not associated with food.

Extracts suitable for use in the present invention may be produced by extraction methods as are known and accepted in the art such as alcoholic extraction, aqueous extractions, carbon dioxide extractions, for example.

As used herein, the term ‘procyanidins’ includes cyanidins, cyanins, procyanins, proanthocyanins, proanthocyanidins, leucoanthocyanins, leucodelphinins, leucocyanins, and anthocyanogens or those similarly envisioned by one of skill in the art. Furthermore, ‘procyanidins’ as disclosed herein belong to a class of flavonoids, found in plants, which are responsible for the brilliant color (red, orange, blue) of fruits and flowers and also have strong antioxidant activity.

Plant Sterols

Plant sterols, or phytosterols, are derived from wood pulp and vegetable oils. Phytosterols are structurally and chemically similar to cholesterol and are unsaturated as they contain one or more double-bonds in their sterol ring group. Differences in the structure between phytosterols and cholesterol result in poor intestinal absorption of phytosterols compared to cholesterol (Lichtenstein A H, Deckelbaum R J. AHA Science Advisory. Stanol/sterol ester-containing foods and blood cholesterol levels. A statement for healthcare professionals from the Nutrition Committee of the Council on Nutrition, Physical Activity, and Metabolism of the American Heart Association. Circulation. 2001 Feb. 27; 103(8):1177-9).

Phytosterols have been shown to be effective at lowering LDL and non-HDL cholesterol (Lau V W, Journoud M, Jones P J. Plant sterols are efficacious in lowering plasma LDL and non-HDL cholesterol in hypercholesterolemic type 2 diabetic and nondiabetic persons. Am J Clin Nutr. 2005 June; 81(6):1351-8) and improving the overall blood lipid profile of humans (Maki K C, Davidson M H, Umporowicz D M, Schaefer E J, Dicklin M R, Ingram K A, Chen S, McNamara J R, Gebhart B W, Ribaya-Mercado J D, Perrone G, Robins S J, Franke W C. Lipid responses to plant-sterol-enriched reduced-fat spreads incorporated into a National Cholesterol Education Program Step I diet. Am J Clin Nutr. 2001 July; 74(1):33-43). This beneficial action of phytosterols is attributed to the ability of the phytosterols to inhibit the intestinal absorption of both dietary and endogenous cholesterol (Normen L, Dutta P, Lia A, Andersson H. Soy sterol esters and beta-sitostanol ester as inhibitors of cholesterol absorption in human small bowel. Am J Clin Nutr. 2000 April; 71(4):908-13).

It is herein understood by the inventors that the incorporation of plant sterols or derivatives of plant sterols in a nutritional composition for improving blood cholesterol levels will effectively inhibit the absorption of cholesterol from ingested food.

In an embodiment of the present invention, which is set forth in greater detail in the examples below, the nutritional composition includes plant sterols or derivatives of plant sterols. A serving of the nutritional composition includes from about 0.03 g to about 1.1 g of plant sterols or derivatives of plant sterols. The preferred dosage of a serving of the nutritional composition comprises about 1.0 g of plant sterols or derivatives of plant sterols.

Plant Stanols

Plant stanols are saturated plant sterols, i.e. they do not contain double-bonds in their sterol ring structures. Plant stanols are typically less abundant in nature than plant sterols (Law M. Plant sterol and stanol margarines and health. BMJ. 2000 Mar. 25; 320(7238):861-4) and likely exert their effects through mechanisms similar to plant sterols.

Plant stanol esters have been shown to reduce total serum cholesterol and LDL levels in hypercholesterolemic men and women (Hallikainen M A, Sarkkinen E S, Uusitupa M I. Plant stanol esters affect serum cholesterol concentrations of hypercholesterolemic men and women in a dose-dependent manner. J Nutr. 2000 April; 130(4):767-76). The LDL-lowering effects of plant stanol esters is fully obtained within one to two weeks of consumption and are sustainable for at least twelve months (Hallikainen M, Sarkkinen E, Wester I, Uusitupa M. Short-term LDL cholesterol-lowering efficacy of plant stanol esters. BMC Cardiovasc Disord. 2002 Aug. 27; 2:14).

It is herein understood by the inventors that the incorporation of plant stanols or derivatives of plant stanols in a nutritional composition for improving blood cholesterol levels will effectively inhibit the absorption of cholesterol.

In an embodiment of the present invention, which is set forth in greater detail in the examples below, the nutritional composition includes plant stanols or derivatives of plant stanols. A serving of the nutritional composition includes from about 0.03 g to about 1.1 g of plant stanols or derivatives of plant stanols. The preferred dosage of a serving of the nutritional composition comprises about 1.0 g of plant stanols or derivatives of plant stanols.

Procyanidins

Procyanidins are the polyphenol pigments responsible for the red, blue and purple colors of plants, including fruits and vegetables. Grape skins are known to be a particularly good source of procyanidins (Del Bas J M, Fernandez-Larrea J, Blay M, Ardevol A, Salvado M J, Arola L, Blade C. Grape seed procyanidins improve atherosclerotic risk index and induce liver CYP7A1 and SHP expression in healthy rats. FASEB J. 2005 March; 19(3):479-81). The polyphenols from grapes used in red wine are believed to be responsible for the coronary health benefits attributed to wine and the ‘French paradox’ (a diet relatively high in fat with a low incidence of coronary disease). This effect has been attributed to the antioxidant properties of these polyphenols. Specifically, the polyphenols found in red wine grapes prevent the oxidation of LDL both in vitro and in vivo (Nigdikar S V, Williams N R, Griffin B A, Howard A N. Consumption of red wine polyphenols reduces the susceptibility of low-density lipoproteins to oxidation in vivo. Am J Clin Nutr. 1998 August; 68(2):258-65). The oxidation of LDL is thought to be a contributing factor to cardiovascular disease (Heinecke J W. Lipoprotein oxidation in cardiovascular disease: chief culprit or innocent bystander? J Exp Med. 2006 Apr. 17; 203(4):813-6).

Red wine polyphenols have been shown to reduce cholesterol while increasing the messenger RNA for HMG-CoA reductase. This has widely been interpreted as a compensatory response instigated by the detection of reduced cholesterol availability (Pal S, Ho N, Santos C, Dubois P, Mamo J, Croft K, Allister E. Red wine polyphenolics increase LDL receptor expression and activity and suppress the secretion of ApoB100 from human HepG2 cells. J Nutr. 2003 March; 133(3):700-6).

It is herein understood by the inventors that the incorporation of procyanidins in a nutritional composition for improving blood cholesterol levels will effectively inhibit the oxidation of LDL and lower cholesterol via its antioxidant activity.

In an embodiment of the present invention, which is set forth in greater detail in the examples below, the nutritional composition includes procyanidins. A serving of the nutritional composition includes from about 0.001 g to about 0.09 g of procyanidins. The preferred dosage of a serving of the nutritional composition comprises about 0.01 g of procyanidins.

Policosanol

Policosanol is a naturally-derived mixture of plant waxes commonly obtained from sugar cane processing. Policosanol possesses cholesterol-lowering activity in both healthy and diabetic humans. Policosanol targets the HMG-CoA reductase enzyme by interfering with its synthesis or degradation. Policosanol has been shown to be a safe and effective lipid-lowering agent compared to accepted medications (Cholesterol-lowering action of policosanol compares well to that of pravastatin and lovastatin. Cardiovasc J S Afr. 2003 May-June; 14(3):161). In addition to lowering LDL levels, policosanol decreases total cholesterol and increases HDL (Janikula M. Policosanol: a new treatment for cardiovascular disease? Altern Med Rev. 2002 June; 7(3):203-17).

It is herein understood by the inventors that the incorporation of policosanol in a nutritional composition for improving blood cholesterol levels will effectively inhibit the synthesis of HMG-CoA reductase as well as enhance its degradation, thereby acting to reduce total blood cholesterol levels.

In an embodiment of the present invention, which is set forth in greater detail in the examples below, the nutritional composition includes policosanol. A serving of the nutritional composition includes from about 0.0005 g to about 0.0075 g of policosanol. The preferred dosage of a serving of the nutritional composition comprises about 0.005 g of policosanol.

Niacin

Niacin, also known as Vitamin B3 or nicotinic acid is one of several water-soluble B-family vitamins. Niacin is often consumed as a nutritional dietary supplement in the form of a multi-vitamin/mineral complex to improve general health. In the United States the RDA (Recommended Daily Allowance) for Niacin is 20 mg, while most commercially available multi-vitamin supplements contain at least 25 mg, and some more than 50 mg.

As a supplement in itself, Niacin has long been successfully used to improve blood lipid profiles (Cheng K, Wu T J, Wu K K, Sturino C, Metters K, Gottesdiener K, Wright S D, Wang Z, O'Neill G, Lai E, Waters M G. Antagonism of the prostaglandin D2 receptor 1 suppresses nicotinic acid-induced vasodilation in mice and humans. Proc Natl Acad Sci USA. 2006 Apr. 25; 103(17):6682-7). Niacin appears to alter lipid levels by inhibiting lipoprotein synthesis and decreasing the production of very low-density lipoproteins (VLDL) particles by the liver (Third report of the National Cholesterol Education Program (NCEP) expert panel on detection, evaluation, and treatment of high blood cholesterol in adults (Adult Treatment Panel III) Final Report. Circ. 2002;106:3143-421).

In a comparative study of 117 individuals, 63 treated with Niacin and 54 treated with a placebo, active treatment resulted in an increase in high-density lipoprotein cholesterol (HDL-C), a decrease in total cholesterol, low-density lipoprotein cholesterol (LDL-C), and triglyceride levels (Squires R W, Allison T G, Gau G T, Miller T D, Kottke B A. Low-dose, time-release nicotinic acid: effects in selected patients with low concentrations of high-density lipoprotein cholesterol. Mayo Clin Proc. 1992 September; 67(9):855-60). Niacin achieves the aforementioned results by reducing lipoprotein synthesis in the liver.

Additionally, niacin is capable of inhibiting the peripheral mobilization of free fatty acids (Grundy S M, Mok H Y, Zech L, Berman M. Influence of nicotinic acid on metabolism of cholesterol and triglycerides in man. J Lipid Res. 1981 January; 22(1):24-36), thereby reducing hepatic secretion of VLDL. Nicotinic acid has been purported as the most effective compound for increasing concentrations of HDL (Vega G L, Grundy S M. Lipoprotein responses to treatment with lovastatin, gemfibrozil, and nicotinic acid in normolipidemic patients with hypoalphalipoproteinemia. Arch Intern Med. 1994 Jan. 10; 154(1);73-82).

It is herein understood by the inventors that the incorporation of Niacin or derivatives of Niacin in a nutritional composition for improving blood cholesterol levels will effectively reduce levels or unhealthy LDL cholesterol and increase levels of healthy HDL cholesterol, by at least the aforementioned mechanisms.

In an embodiment of the present invention, which is set forth in greater detail in the examples below, the nutritional composition includes niacin. A serving of the nutritional composition includes from about 0.01 g to about 0.07 g of niacin. The preferred dosage of a serving of the nutritional composition comprises about 0.017 g of niacin.

Xanthinol Nicotinate

Xanthinol nicotinate is one of several forms of Niacin (vitamin B3). It easily passes through the cell membrane and is considered the most potent form of Niacin. Pharmaceutically, Xanthinol nicotinate is classified as a vasodilator.

In patients with peripheral arterial obliterative disease, Xanthinol nicotinate was found to have anti-platelet and thrombolytic actions (Bieron K, Swies J, Kostka-Trabka E, Gryglewski R J. Thrombolytic and antiplatelet action of xanthinol nicotinate (Sadamin): possible mechanisms. J Physiol Pharmacol. 1998 June; 49(2):241-9).

It is herein understood by the inventors that the incorporation of Xanthinol nicotinate, as a derivative of niacin, in a nutritional composition for improving blood cholesterol levels will effectively reduce levels or unhealthy LDL cholesterol and increase levels of healthy HDL cholesterol, by at least the aforementioned mechanisms.

In an embodiment of the present invention, which is set forth in greater detail in the examples below, the nutritional composition includes xanthinol nicotinate. A serving of the nutritional composition includes from about 0.01 g to about 0.07 g of xanthinol nicotinate. The preferred dosage of a serving of the nutritional composition comprises about 0.05 g of xanthinol nicotinate.

In a preferred embodiment of the present invention, the composition is comprised of a source of an effective amount of plant sterols or derivatives of plant sterols, a source of an effective amount of procyanidins, a source of an effective amount of policosanol and niacin or derivatives of niacin.

In another embodiment of the present invention, the composition is comprised of a source of an effective amount of plant stanols or derivatives of plant stanols, a source of an effective amount of procyanidins, a source of an effective amount of policosanol and niacin or derivatives of niacin.

Not wishing to be bound by theory, it is believed that the nutritional composition of the present invention will act substantially simultaneously to improve blood cholesterol levels by affecting multiple, non-mutually exclusive mechanisms. Therapeutically effective amounts of plant sterols or plant stanols or derivatives thereof will inhibit the absorption of cholesterol; the antioxidant activity of therapeutically effective amounts of procyanidins will inhibit the oxidation of LDL and lower cholesterol; policosanol in therapeutically effective amounts will lower cholesterol by affecting the synthesis or degradation of HMG-CoA reductase; niacin or derivatives of niacin in therapeutically effective amounts will lower cholesterol by inhibiting the peripheral mobilization of free fatty acids, thereby reducing hepatic secretion of VLDL. Additionally, therapeutically effective amounts of niacin or derivatives of niacin will act to increase concentrations of HDL in the body.

Additional embodiments of the present invention may also include portions of the composition as fine-milled ingredients. U.S. Non-Provisional patent application Ser. No. 11/709,526 entitled “Method for Increasing the Rate and Consistency of Bioavailability of Supplemental Dietary Ingredients” filed Feb. 21, 2007, which is herein fully incorporated by reference, discloses a method of increasing the rate of bioavailability following oral administration of components comprising supplemental dietary compositions by the process of particle-milling.

For the purposes of the present invention, the terms micronization, milling, particle-milling, and fine-milling are used interchangeably, wherein they refer to a technology, process and end-products involved in or leading to a narrowing of particle size range and a concomitant reduction in the average particle size. For the purposes of the present invention, acceptable milled-particle sizes are in the range of from about 1 nanometer to about 500 microns.

Further to improving bioavailability, it is understood by the inventors that increased solubility resulting from fine-milling will lead to improvements in characteristics in which solubility and reduced particle size likely play a role.

Furthermore, additional embodiments of the present invention may be incorporated into specific controlled-release solid dosage forms. U.S. Non-Provisional patent application Ser. No. 11/709,525 entitled “Method for a Supplemental Dietary Composition Having a Multi-Phase Dissolution Profile” filed Feb. 21, 2007, which is herein fully incorporated by reference, discloses a method of achieving a solid oral dosage form with multiple dissolution characteristics for the release of active ingredients. Conventional oral dosage formulations are bound by the rate of dissolution of the unprocessed substance, thereby limiting the rate of bioavailability of the substance upon oral administration. This is particularly problematic for poorly-soluble compounds which have an inherently low rate of dissolution in that they may be excreted prior to first-pass.

It is herein understood that, due to the relationship between solubility and dissolution, the amount of a substance in solution at any given time is dependent upon both dissolution and solubility. Furthermore, it is understood by way of extension that increasing the rate of dissolution of a given substance acts to reduce the time to dissolution of a given solute or substance in a given solvent. However, the absolute solubility of said solute does not increase with infinite time. Thus, increasing the rate of dissolution of a substance will increase the amount of said substance in solution at earlier points in time, thus increasing the rate of bioavailability of said substance at earlier times upon oral administration.

The increase in the rate of bioavailability will allow better and quicker compound transfer to the systemic parts of the body.

Micronization is a technique which has been used as a method of sizing solid compounds to fine powders. Following a micronization process, compounds and more specifically poorly soluble compounds are transformed into fine powders which can then be transformed into suitable, stable and patient-compliant dosage forms. These forms, for the purposes of the present invention are derived for oral administration.

Micronization techniques offer an advantage over larger forms of compounds and poorly soluble compounds—following micronization, compounds have higher surface area to volume ratio. This provides for, as compared to physically coarse compounds, an ultrafine micronized powder that has a significantly increased total surface area. Mathematically, cross-sectional surface area increases with the square of the radius, while volume increases with the cube of the radius. Therefore, as a particle becomes smaller, the volume of the particle decreases at a faster rate than the surface area leading to an increase in the ratio of surface area to volume. By way of theoretical calculations, decreasing the size of a particle can increase its rate of dissolution via increasing the surface area to volume ratio. In the case of solubility, this increase in relative surface area allows for greater interaction with solvent. Additional embodiments of the present invention may employ a multi-phasic dissolution profile to provide a time-release mechanism.

According to various embodiments of the present invention, the nutritional supplement may be consumed in any form. For instance, the dosage form of the nutritional supplement may be provided as, e.g., a powder beverage mix, a liquid beverage, a ready-to-eat bar or drink product, a capsule, a liquid capsule, a tablet, a caplet, or as a dietary gel. The preferred dosage forms of the present invention are as a caplet or as a liquid capsule.

Furthermore, the dosage form of the nutritional supplement may be provided in accordance with customary processing techniques for herbal and nutritional supplements in any of the forms mentioned above. Additionally, the nutritional supplement set forth in the example embodiment herein may contain any appropriate number and type of excipients, as is well known in the art.

The present nutritional composition or those similarly envisioned by one of skill in the art, may be utilized in methods to improve blood cholesterol levels in a formulation designed to be consumed on a daily basis.

Although the following examples illustrate the practice of the present invention in four of its embodiments, the examples should not be construed as limiting the scope of the invention. Other embodiments will be apparent to one of skill in the art from consideration of the specifications and example.

### EXAMPLES

### Example 1

A nutritional composition is provided in two servings per day as caplets. A single serving of the nutritional composition comprises from about 0.30 g to about 1.10 g of plant sterol esters, about 0.005 g to about 0.45 g of grape skin extract standardized for 20% proanthocyanidins, about 0.0005 g to about 0.0075 g of policosanol, and about 0.01 g to about 0.07 g of Xanthinol nicotinate.

Directions: As a diet supplement, 2 caplets are administered with an 8 oz. glass of water two (2) times daily. Each two caplets or liquid capsules serving may be consumed approximately 30 to 60 minutes before meals.

### Example 2

A nutritional composition is provided in two servings per day as caplets. A single serving of the nutritional composition comprises from about 0.30 g to about 1.10 g of plant sterol esters, about 0.005 g to about 0.45 g of grape skin extract standardized for 20% proanthocyanidins, about 0.0005 g to about 0.0075 g of policosanol, about 0.01 g to about 0.07 g of Niacin, about 0.005 g to about 0.05 g of pectin, about 0.0005 g to about 0.0075 g of cocoa polyphenols and about 0.0005 g to about 0.0030 g of citrus flavonoids.

Directions: As a diet supplement, 2 caplets are administered with an 8 oz. glass of water two (2) times daily. Each two caplets or liquid capsules serving may be consumed approximately 30 to 60 minutes before meals.

### Example 3

A nutritional composition is provided in two servings per day as caplets. A single serving of the nutritional composition comprises about 1.0 g plant sterols, about 0.05 g grape skin extract standardized for 20% proanthocyanidins, about 0.005 g of policosanol and about 0.05 g of Xanthinol nicotinate.

Directions: As a diet supplement, 2 caplets are administered with an 8 oz. glass of water two (2) times daily. Each two caplets or liquid capsules serving may be consumed approximately 30 to 60 minutes before meals.

### Example 4

A nutritional composition is provided in two servings per day as caplets. A single serving of the nutritional composition comprises about 1.0 g plant sterols, about 0.05 g grape skin extract standardized for 20% proanthocyanidins, about 0.005 g of policosanol and about 0.017 g of Niacin.

Directions: As a diet supplement, 2 caplets are administered with an 8 oz. glass of water two (2) times daily. Each two caplets or liquid capsules serving may be consumed approximately 30 to 60 minutes before meals.

### Extensions and Alternatives

In the foregoing specification, the invention has been described with a specific embodiment thereof; however, it will be evident that various modifications and changes may be made thereto without departing from the broader spirit and scope of the invention.

What is claimed:

1. A composition for improving blood cholesterol levels in an individual comprising:
from about 0.30 g to about 1.100 g of plant sterols or derivatives of plant sterols;
from about 0.001 g to about 0.090 g of procyanidins;
from about 0.0005 g to about 0.0075 g of policosanol; and
from about 0.010 g to about 0.070 g of niacin or derivatives of niacin.

2. The composition of claim 1, wherein the amount of the plant sterols or derivatives of plant sterols is about 1.0 g;
the amount of the procyanidins is about 0.01 g;
the amount of the policosanol is about 0.005 g; and
the amount of niacin or derivatives of niacin is about 0.05 g.

3. The composition of claim 1, wherein;
the plant sterols or derivatives of plant sterols, the procyanidin, the policosanol, and the niacin or derivatives of niacin act substantially simultaneously to inhibit cholesterol absorption, decrease total blood cholesterol levels, decrease blood LDL levels, increase blood HDL levels and interfere with HMG-CoA reductase activity.

4. The composition of claim 1, wherein at least a portion of one or more of the ingredients is fine-milled.

5. The composition of claim 1, wherein the plant sterols or derivatives of plant sterols, the procyanidin, the policosanol, and the niacin or derivatives of niacin comprise an oral dosage form having a multi-phasic rate of dissolution.

6. The composition of claim 5, wherein said multi-phasic rate of dissolution comprises a first-phase and a second-phase; whereby said first-phase has a first rate of dissolution said second-phase has a second rate of dissolution.

7. The composition of claim 6, further comprising a third-phase, whereby said third-phase has a third rate of dissolution.

8. The composition of claim 6, wherein the multi-phasic rate of dissolution provides a time-release mechanism.

9. A method for improving blood cholesterol levels in an individual comprising the step of orally administering to the individual a composition comprising:
from about 0.30 g to about 1.100 g of plant sterols or derivatives of plant sterols;
from about 0.001 g to about 0.090 g of procyanidins;
from about 0.0005 g to about 0.0075 g of policosanol; and
from about 0.010 g to about 0.070 g of niacin or derivatives of niacin.

10. The method of claim 9, wherein the composition is administered at least twice daily.

11. The method of claim 10, wherein the composition is administered prior to meals.

12. A composition for improving blood cholesterol levels in an individual comprising:
from about 0.30 g to about 1.100 g of plant stanols or derivatives of plant stanols;
from about 0.001 g to about 0.090 g of procyanidins;
from about 0.0005 g to about 0.0075 g of policosanol; and
from about 0.010 g to about 0.0700 g of niacin or derivatives of niacin.

13. The composition of claim 12, wherein the amount of the plant stanols or derivatives of plant stanols is about 1.0 g;
the amount of the procyanidins is about 0.01 g;
the amount of the policosanol is about 0.005 g; and
the amount of niacin or derivatives of niacin is about 0.05 g.

14. The composition of claim 12, wherein;
the plant stanols or derivatives of plant stanols, the procyanidin, the policosanol, and the niacin or derivatives of niacin act substantially simultaneously to inhibit cholesterol absorption, decrease total blood cholesterol levels, decrease blood LDL levels, increase blood HDL levels and interfere with HMG-CoA reductase activity.

15. The composition of claim 12, wherein at least a portion of one or more ingredients is fine-milled.

16. The composition of claim 12, wherein the plant stanols or derivatives of plant stanols, the procyanidin, the policosanol, and the niacin or derivatives of niacin comprise an oral dosage form having a multi-phasic rate of dissolution.

17. The composition of claim 12, wherein said multi-phasic rate of dissolution comprises a first-phase and a second-phase; whereby said first-phase has a first rate of dissolution said second-phase has a second rate of dissolution.

18. The composition of claim 18, further comprising a third-phase, whereby said third-phase has a third rate of dissolution.

19. The composition of claim 18, wherein the multi-phasic rate of dissolution provides a time-release mechanism.

20. A method for improving blood cholesterol levels in an individual comprising the step of orally administering to the individual a composition comprising:
from about 0.30 g to about 1.100 g of plant stanols or derivatives of plant stanols;
from about 0.001 g to about 0.090 g of procyanidins;
from about 0.0005 g to about 0.0075 g of policosanol; and
from about 0.010 g to about 0.070 g of niacin or derivatives of niacin.

21. The method of claim 20, wherein the composition is administered at least twice daily.

22. The method of claim 21, wherein the composition is administered prior to meals.
